(19) United States
(12) Patent Application Publication
(10) Pub. No.: US 2004/0168215 A1
(43) Pub. Date: Aug. 26, 2004

(54) Plant gene expression system for processing, targeting and accumulating foreign proteins in transgenic seeds

(76) Inventors: Liwen Jiang, Hong Kong (CN); Samuel Sai Ming Sun, Hong Kong (CN)

Correspondence Address: MCANDREWS HELD & MALLOY, LTD, 500 WEST MADISON STREET, SUITE 3400, CHICAGO, IL, 60661

(21) Appl. No.: 10/783,620

(22) Filed: Feb. 20, 2004

Related U.S. Application Data
(60) Provisional application No. 60/449,367, filed on Feb. 21, 2003.

(51) Int. Cl. A01H 1/00; C12N 15/82; C07H 21/04

(52) U.S. Cl. 800/287; 536/23.6; 435/468

(57) ABSTRACT

A DNA construct generates and directs the processing, targeting and stable accumulation of target proteins in transgenic plant seeds. A method for constructing transgenic plants provides a general strategy in which unique transmembrane domain and cytoplasmic tail sequences are used as anchors for delivering recombinant target proteins via distinct vesicular transport pathways to specific vacuolar compartments, thus enabling stable accumulation of foreign target proteins in transgenic plants. A plant gene expression system has flexibility to allow the target proteins to bypass or subject to plants Golgi-specific post-translational modifications.

### CROSS REFERENCE OF RELATED APPLICATIONS

The present application claims the benefit of U.S. provisional application Serial No. 60/449,367 filed on Feb. 21, 2003, entitled the same, now pending, which is explicitly incorporated herein by reference in its entirety.

### BACKGROUND OF THE INVENTION

1. Field of the Invention

The present invention is directed to a gene expression system, particularly to a plant gene expression system for processing, targeting and accumulating foreign proteins in transgenic seeds.

2. Description of Prior Art

Using transgenic plants as general expression systems and bioreactors is an attractive and competitive approach for the economic production of pharmaceutical recombinant proteins and enzymes for industrial use. Recombinant proteins expressed in plant cells are subjected to post-translational modification when they enter the secretory pathway, which could represent a major limitation for the expression of recombinant glycoproteins of mammalian origin. The subcellular localization of recombinant proteins expressed in plant cells not only affects the stability of protein structure and accumulation, but also determines the efficiencies of protein recovery and purification. Several plant expression systems have been tested for their suitability for protein expression and production, including seed oil-bodies, root exudates, phyllosecretion (guttation fluid), cell suspension culture and transgenic plants (Borisjuk, N. V. et al. (1999), Production of recombinant proteins in plant root exudates, Nat. Biotechnol., 17, 466-469; Komarnytsky, S. et al. (2000), Production of recombinant proteins in tobacco guttation fluid, Plant Physiol., 124, 927-933; Conrad, U. and Fiedler, U. (1998), Compartment-specific accumulation of recombinant immunoglobulins in plant cells: an essential tool for antibody production of physiological functions and pathogen activity, Plant Mol. Biol., 38, 101-109; Giddings, G. et al. (2000), Transgenic plants as factories for biopharmaceuticals, Nat. Biotech. 18, 1151-1155; Fisher, R. and Emans, N. (2000), Molecular farming of pharmaceutical proteins, Transgenic Res., 9, 279-299).

More recent examples include inducible expression of cellulase in chloroplasts of transgenic plants and accumulation of recombinant proteins in the endoplasmic reticulum (ER) (Heifetz, P. B. and Tuttle, A. M. (2001), Protein expression in plastids, Curr. Opin. Plant Biol., 4, 157-161; Scheller, J. et al. (2001), Production of spider silk proteins in tobacco and potato, Nature Biotechnol., 19, 573-577). In these systems, proteins are targeted to various compartments, such as cytosol, chloroplast, ER or ER-derived protein bodies, oil bodies and apoplast, where they can stably accumulate. Although there has been some degree of success, the yield of recombinant protein production in most experimental systems has been low. For example, expression of recombinant antigens in transgenic plants ranges from only 0.01 to 1% of total soluble proteins. Such low accumulation of recombinant proteins might be because of low expression or, more likely, because the proteins expressed are targeted for degradation by the proteolytic systems of the plant, and are consequently unstable and have a high turnover rate.

One major difference between plant cells and those of yeast and mammals is that plant cells store many types of metabolic products in vacuoles, including proteins. It has long been known that plant vacuoles can perform multiple functions in plant cells, such as storage, digestion and growth (Wink, M. (1993), The plant vacuole: a multifunctional compartment, J. Exp. Bot., 44, 231-146). In contrast to mammalian and yeast cells in which a single lysosome and/or vacuole functions as a degradative or lytic compartment, plant cells contain both lytic vacuoles and protein storage vacuoles (PSVs), which are separate organelles with distinct functions, and vacuolar compartments receive their contents via different vesicular transport pathways (Neuhaus, J. M. and Rogers, J. C. (1998), Sorting of proteins to vacuoles in plant cells, Plant Mol. Biol., 38, 127-144; Raikhel, N. V. and Vitale, A. (1999), What do proteins need to reach different vacuoles? Trends Plant Sci., 4, 149-155; Robinson, D. G. et al. (2000), Post-Golgi prevacuolar compartments, Ann. Plant Rev., 5, 270-298). Thus, optimal storage of proteins or metabolites in plants requires delivery of the product to the correct type of vacuole where proteins can undergo stable accumulation. For example, when the seed storage protein vicilin was expressed and targeted to the lytic vacuoles in vegetative tissues of transgenic plants, no detectable amount of protein was obtained (Wandelt, C. I. et al. (1992), vicilin with carboxy-terminal KDEL is retained in the endoplasmic reticulum and accumulates to high levels in the leaves of transgenic plants, Plant J., 2, 181-192). However, the addition of a KDEL sequence to its C-terminal, which would allow retention of vicilin within the ER, thus keeping it from the digestive environment within the lytic vacuole, resulted in the accumulation of vicilin in leaves, where it accounted for 1% of extractable proteins. By contrast, seeds are generally rich in proteins and a large percentage of the soluble proteins (defined as lumenal proteins without membrane attachments) are stored in the PSVs. The PSV is therefore an ideal compartment for storage of various foreign recombinant proteins. Additionally, the PSV in most seeds is a compound organelle with three unique subcompartments (the matrix, the globoid and the crystalloid) (Jiang, L. et al. (2000), Biogenesis of the protein storage vacuole crystalloid, J Cell Biol., 150, 755-769; Jiang, L. and Rogers, J. C. (2001), Compartmentation of proteins in the protein storage vacuole of plant cells, Adv. Bot. Res., 35, 163-197), which would provide different environments and functions within the PSV. Recent evidence indicates that PSVs may contain proteases that are activated for the process of storage proteins within the PSV germinating seeds (Toyooka, K. et al. (2000), Mass transport of proform of a KDEL-tailed cysteine protease (SH-EP) to protein storage vacuoles by ER-derived vesicles is involved in protein mobilization in germinating seeds, J Cell Biol., 148, 453-464; Herman, E. M. and Larkins, B. A. (1999), Protein storage bodies, Plant Cell, 11, 601-613), and that the PSV globoid might function as a lytic vacuole (Jiang, L. et al. (2000), Biogenesis of the protein storage vacuole crystalloid, J Cell Biol., 150, 755-769; Jiang, L. and Rogers, J. C. (2001), Compartmentation of proteins in the protein storage vacuole of plant cells, Adv. Bot. Res., 35, 163-197).

Soluble proteins that are destined for plant vacuoles contain positive targeting information that causes them to be sorted away from the flow of proteins to be transported outside the cell. Three general types of vacuolar sorting determinants have been described in plant proteins, including the N-terminal determinants of sporamin and aleurain, C-terminal determinants of phaseolin and albumin, and the internal sorting determinant of ricin (Neuhaus, J. M. and Rogers, J. C. (1998), Sorting of proteins to vacuoles in plant cells, Plant Mol. Biol., 38, 127-144; Raikhel, N. V. and Vitale, A. (1999), What do proteins need to reach different vacuoles? Trends Plant Sci., 4, 149-155; Matsuoka, K. and Neuhaus, J. M. (1999), Cis-elements of protein transport to the plant vacuoles, J. Exp. Bot., 50, 165-174; Frigerio, L. et al. (2001), The internal propeptide of the ricin precursor carries a sequence-specific determinant for vacuolar sorting, Plant Physiol., 126, 167-175). In contrast to protein sorting to the lysosome in mammalian cells where glycosylation of the targeted proteins in the Golgi is required for lysosomal targeting (Braulke, T. (1996), Origin of lysosomal proteins, Subcellular Biochem, 27, 15-49; Griffiths, G. B. et al. (1988), The mannose 6-phosphate receptor and the biogenesis of lysosome, Cell, 52, 329-341), studies of plant proteins thus far indicate that glycosylation of the targeted proteins is not required for either vacuolar targeting or extracellular secretion (Voelker, T. A. et al. (1989), In vitro mutated phytohemagglutinin genes expressed in tobacco seeds: role of glycans in protein targeting and stability, Plant Cell, 1, 95-104; Lerouge, P. et al. (1996), N-linked oligosaccharide processing is not necessary for glycoprotein secretion in plants, Plant J., 10, 713-719). In addition, soluble proteins can also reach vacuoles or protein bodies via different mechanisms. For example, in cereals such as rice and wheat, ER-derived protein bodies are responsible for the deposition and accumulation of prolamins in PSV, whereas glutelins reach PSV via a Golgi-mediated pathway (Okita, T. W. and Rogers, J. C. (1996), Compartmentation of proteins in the endomembrane system of plant cells, Ann Rev. Plant Physiol. Plant Mol. Biol., 47, 327-350; Galili, G. et al. (1998), The endoplasmic reticulum of plant cells and its role in protein maturation and biogenesis of oil bodies, Plant Mol. Biol., 38, 1-29). Interestingly, overexpression of certain seed storage proteins in transgenic plants induces cells to produce new vesicles in either vegetative cells or seeds, which could serve as intermediate storage compartments where expressed proteins can be stably accumulated because these inducible organelles are kept separated from the proteolytic vacuolar environment (Hayashi, M. et al. (1999), Accumulation of a fusion protein containing 2S albumin induces novel vesicles in vegetative cells of Arabidopsis, Plant Cell Physiol., 40, 263-272; Kinnery, A. J. et al. (2001), Cosuppression of the α subunits of β-conglycinin in transgenic soybean seeds induces the formation of endoplasmic reticulum-derived protein bodies, Plant Cell, 13, 1165-1178). Thus, these inducible vesicles might be one of the compartments that could be used as storage organelles for accumulating recombinant proteins in transgenic plants.

Multiple vesicular transport pathways are involved in sorting soluble proteins to vacuoles. Protein sorting to the lytic vacuole is a receptor-mediated process that involves BP-80 and its homologues, a type I integral membrane protein that belongs to a family of vacuolar sorting receptor (VSR) proteins (Paris, N. et al. (1997), Molecular cloning and further characterization of a probable plant vacuolar sorting receptor, Plant Physiol., 115, 29-39; Jiang, L. and Rogers, J. C. (1998), Integral membrane protein sorting to vacuoles in plant cells: evidence for two pathways, J. Cell Biol., 143, 1183-1199; Ahmed, S. U. et al. (1997), Cloning and subcellular location of an Arabidopsis receptor-like protein that shares common features with protein-sorting receptors of eukaryotic cells, Plant Physiol., 114, 325-336; Shimada, T. et al. (1997), A pumpkin 72-kDa membrane protein of precursor-accumulating vesicles has characteristics of a vacuolar sorting receptor, Plant Cell Physiol., 38, 1414-1420). In yeast, it appears that the vacuole is the default destination for integral membrane proteins (Roberts, C. J. et al. (1992), Membrane protein sorting in the yeast secretory pathway: evidence that the vacuole may be the default compartment, J. Cell Biol., 119, 69-83). By contrast, the sorting of integral membrane proteins to specific vacuoles in plant cells requires specific sequences derived from the transmembrane domain (TMD) and cytoplasmic tail (CT) (Jiang, L. et al. (2000), Biogenesis of the protein storage vacuole crystalloid, J Cell Biol., 150, 755-769; Frigerio, L. et al. (2001), The internal propeptide of the ricin precursor carries a sequence-specific determinant for vacuolar sorting, Plant Physiol., 126, 167-175; Jiang, L. and Rogers, J. C. (1999), Functional analysis of a plant Kex2p protease in tobacco suspension culture cells, Plant J., 18, 23-32; Hofte, H. and Chrispeels, M. J. (1992), Protein sorting to the vacuolar membrane, Plant Cell, 4, 995-1004). Thus, three vesicular pathways are marked by traffic of three integral membrane reporter proteins that contain specific TMD and CT sequences (Jiang, L. et al. (2000), Biogenesis of the protein storage vacuole crystalloid, J Cell Biol., 150, 755-769; Jiang, L. and Rogers, J. C. (1998), Integral membrane protein sorting to vacuoles in plant cells: evidence for two pathways, J. Cell Biol., 143, 1183-1199; Jiang, L. and Rogers, J. C. (1999), Sorting of membrane proteins to vacuoles in plant cell, Plant Sci., 146, 55-67). For example, a reporter containing the BP-80 TMD and CT reached the lytic vacuole via the Golgi, whereas substitution with the α-tonoplast intrinsic protein (α-TIP) CT redirected the reporter to the PSV, bypassing the Golgi (Jiang, L. and Rogers, J. C. (1998), Integral membrane protein sorting to vacuoles in plant cells: evidence for two pathways, J. Cell Biol., 143, 1183-1199). The study provides the first demonstration that specific TMD and CT sequences can be used to direct reporter proteins to specific vacuolar compartments via different vesicular pathways in plant cells. Similarly, when a membrane-anchored yeast invertase was expressed in transgenic plants, this protein was targeted to the vacuole via the Golgi (Barrieu, F. and Chrispeel, M. J. (1999), Delivery of a secreted soluble protein to the vacuole via a membrane anchor, Plant Physiol., 120, 961-968).

Taking advantages of the understanding of trafficking and targeting of storage protein to specific subcompartments within the seed PSV, the present invention provides methods for stable and optimal accumulation of foreign target proteins in transgenic seeds. The approach described here, would allow a level of protein accumulation within the PSVs that could be as much as 8-10% of total seed proteins, as demonstrated by reporter proteins using confocal immunofluorescence and by expressing the Lysine-rich protein expressed in transgenic seeds.

### SUMMARY OF THE INVENTION

Therefore, one object of the invention is to provide a DNA construct to generate and direct the processing, targeting and stable accumulation of a target protein in transgenic plant seeds. The DNA construct in turn comprises:

a promoter sequence capable of directing expression in plant seed cells;

a first DNA sequence encoding the target protein;

a second DNA sequence having transmembrane domain (TMD) and cytoplasmic tail (CT) sequences serving as anchors for delivering recombinant target proteins via distinct vesicular transport pathways to specific vacuolar compartments; and

a third DNA sequence functioning as a termination region in the plant.

In one embodiment of the invention, the DNA construct further comprises a spacer sequence in front of the TMD sequence so that the membrane anchorage does not affect the structure of the protein and proper protein folding can occur. Preferably, the spacer sequence is a proteolytic cleavage sequence.

In another embodiment of the invention, the DNA construct may further comprise an engineered signal peptide sequence if the recombinant protein does not contain a predicted signal sequence that functions in the plant cells. The signal peptide sequence may be derived from proaleurain.

The promoter used in the DNA construct is preferably a seed-specific promoter such as a phaseolin promoter.

In a preferred embodiment of the invention, the TMD sequence may be derived from BP-80, and the CT sequence is derived from BP-80 or α-TIP.

The third DNA sequence functioning as a termination region in the invention may be an NOS terminator.

In the invention, the target proteins can be of diverse origins, such as those proteases or proteins resistant to acidified environment, and also can be one that would favor their stable accumulation, correspondingly. The vacuolar compartments can be seed protein storage vacuoles (PSVs) and their subcompartments or vacuoles in vegetative tissues. Preferably, the protein storage vacuoles and their subcompartments provide a protease activity acting with the proteolytic cleavage sequence so that the target protein can separate from the transmembrane domain.

In the present invention, the target proteins may possess biological or pharmaceutical functions and can be applied for industry uses.

Another object of the invention is to provide an expression system in transgenic plants seeds for enhancing target proteins production with flexibility for the target proteins to bypass or acquire post-translational modifications. The expression system comprises a vector into which is inserted a DNA construct as defined above.

In an embodiment of the expression system of the invention, the target proteins can be devoid of the post-translational modification through bypassing the Golgi modification, wherein the post-translational modification can be glycosylation of the target proteins.

It is also an object of the present invention to provide an expression system for enhanced protein production through stable accumulation of these target proteins in transgenic plants' seeds. It is another object of the present invention to provide flexibility for the target proteins to acquire or to bypass plant Golgi-specific post-translational modifications.

The invention also provides a host cell comprising a DNA construct as defined herein. The host cell is preferably a plant cell and the plant may be selected from monocots and dicots.

The invention still provides a transgenic plant or progeny thereof comprising a DNA construct as defined herein and edible parts of the transgenic plant or progeny thereof defined herein.

Yet another object of the invention is to provide a transgenic plant seed and a transgenic plant culture cell that comprises a DNA construct as defined herein.

Still another object of the invention is to provide propagation materials of the transgenic plant or progeny thereof or plant cell defined herein.

Food and food supplements generated from the transgenic plant or progeny thereof or plant cells or plant seeds as defined herein are also the objects of the invention.

Still another object of the invention is to provide a method for constructing a transgenic plant comprising transgenic plant seeds expressing target proteins. The method comprises the steps of:

a) constructing a vector including a DNA construct defined herein;

b) transforming plant cells with the vector; and

c) regenerating the transgenic plant from the plant cells to produce the target protein in the plant seeds.

In one embodiment of the method according to the invention, the plant cells are transformed utilizing an Agrobacterium system, such as an Agrobacterium tumefaciens-Ti plasmid system.

In the method of the invention, the vector used may be a plasmid vector such as a superbinary vector, preferably pSB130, or a binary vector, preferably pBI121.

Another object of the invention is to provide a use of the DNA construct as defined herein for processing, targeting and stable accumulation of target protein in transgenic plant seeds. The target proteins possess biological or pharmaceutical functions and can be applied for industry uses.

### BRIEF DESCRIPTION OF THE DRAWINGS

FIG. 1 shows a chimeric construct for targeting recombinant proteins to PSV globoid subcompartment via Golgi according to the invention.

FIG. 2 shows a chimeric construct for targeting recombinant proteins to PSV crystalloid subcompartment from ER directly according to the invention.

FIG. 3 shows the coding regions of the four chimeric constructs A, B, C and D that are used for plant transformation and subsequent analysis, in which abbreviations mean: sp, signal peptide; YFP, yellow fluorescent protein; TMD, transmembrane domain from BP-80; CT, cytoplasmic tail; PSV, protein storage vacuole; hG-CSF, human granulocyte-colony stimulating factor; and POL, Polygonatum odoratum lectin.

FIG. 4 shows the Western blot analysis of transgenic tobacco plants expressing construct A or B (FIG. 3). Soluble (CS) and membrane (CM) proteins were extracted from leaves of both transgenic plants expressing construct A or B and from wild type plants, followed by SDS-PAGE and Western blot detection using anti-GFP antibody, and the full-length fusion protein and the cleaved YFP protein were indicated by double asterisks and single asterisk, respectively.

FIG. 5 shows the Western blot analysis of transgenic tobacco seeds. Soluble (CS) and membrane (CM) proteins were extracted from seeds of transgenic expressing construct A or B (FIG. 3) and from wild type plants, followed by SDS-PAGE and Western blot detection using anti-GFP antibody, and The cleaved soluble YFP protein was indicated by asterisk.

FIG. 6 shows the subcellular localization of YFP fusions in transgenic seeds. Fresh sections were prepared from developing transgenic seeds expressing construct A or B (FIG. 3), followed by directly observation for YFP signals using confocal laser scanning microscope. Shown are YFP signals from the expressed proteins and the DIC (differential interface contrast) images of the observed cells and the Merged of the two.

FIG. 7 shows the Western blot analysis of hG-CSF fusion (FIG. 3) in transgenic rice seeds. Soluble (CS) and membrane (CM) proteins were extracted from mature seeds of three individual transgenic rice expressing construct C, followed by SDS-PAGE and western blot detection with anti-hG-CSF or anti-BP-80 CT antibodies. Double asterisks indicate the position of the intact hG-CSF fusion protein.

FIG. 8 shows the Western blot analysis of POL fusion (FIG. 3) in transgenic rice seeds. Soluble (CS) and membrane (CM) proteins were extracted from mature seeds of two individual transgenic rice expressing construct D, followed by SDS-PAGE and Western blot detection with anti-BP-80 CT or anti-POL antibodies. Double asterisks indicate the position of the intact POL fusion protein.

### DETAILED DESCRIPTION OF THE PREFERRED EMBODIMENTS

As described before, one aspect of the invention is achieved by stable accumulation of foreign target protein in transgenic seeds through application of transmembrane and cytoplasmic tail sequence as anchors, for delivering recombinant target proteins via distinct vesicular transport pathways to specific vacuolar compartments, thereby providing flexibility for the target protein to acquire plant Golgi-specific post-translational modifications.

Several organelles within the plant cells might serve as places for targeting and storing recombinant proteins in transgenic plants. Seed protein storage vacuoles (PSVs) are particularly attractive for such a purpose because PSVs accumulate abundant proteins derived from either the Golgi or from ER directly. The present invention provides a method through manipulating specific membrane sequences to target recombinant proteins to the PSVs via either of these routes, which would allow flexibility for the recombinant protein to acquire or bypass Golgi-modification. Although plant-specific Golgi-modification would be a major limitation to the manufacture of glycoproteins of mammalian origin, the method disclosed below would allow recombinant proteins to bypass the Golgi apparatus on the way to PSVs, an attractive and simple means of delivering large quantities of foreign recombinant proteins to specific vacuolar compartments.

Using plant seeds as bioreactors for the production of recombinant proteins is an attractive approach because seeds can be stored for a long period, conveniently transported and consumed directly. Taking advantage of specific vacuolar compartmentization of proteins via different vesicular pathways in plant cells, particularly in plant seeds, the present invention makes use of several plant organelles and compartments as potential targets for transporting and accumulating soluble recombinant proteins in transgenic plants, including ER, ER-derived vesicles, chloroplasts, vacuoles and the apoplast.

1) Seed-Specific Promoters

The present invention takes further advantage of the strong expression of seed storage proteins in plants particularly in plant seeds. For example, a seed-specific protein phaseolin may be used for constructing a chimeric gene to transform plants to produce the target proteins. As phaseolin is an abundant seed protein, the phaseolin promoter, which is seed specific, is of great significance for transgenic expression of foreign proteins. Alternative promoters known to the skilled person may also be used, provided that they have the ability to efficiently produce foreign proteins, particularly in plant seeds.

2) Expression Cassettes

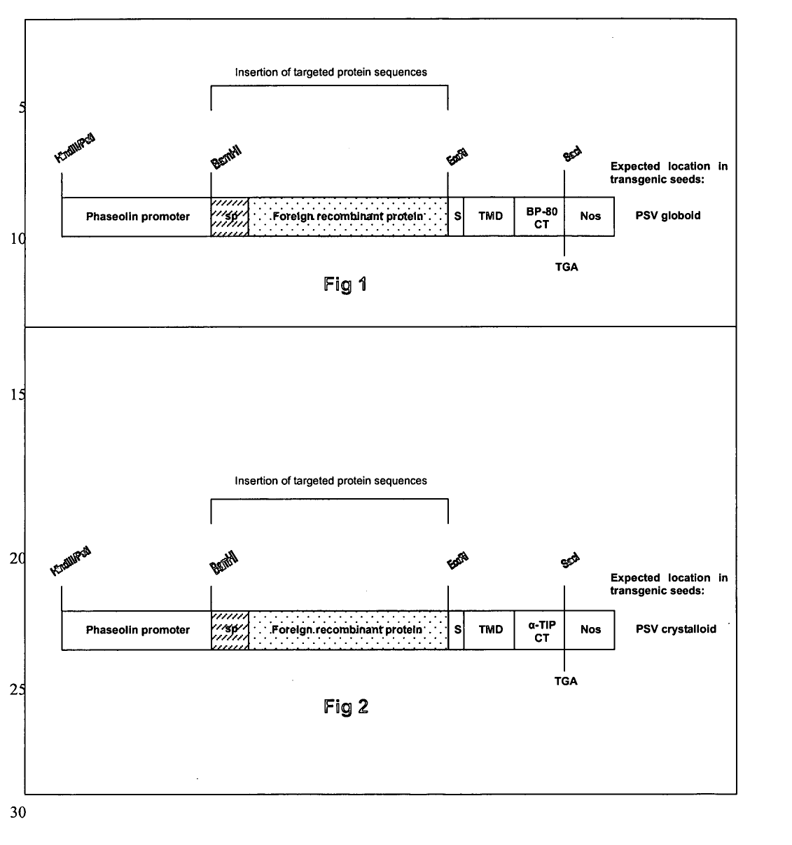

Based on appropriate promoter selection and understanding of traffic of integral membrane proteins to vacuoles in plant cells using both in vitro and in vivo systems, the present invention suggests a method using expression cassettes for translational fusion with a combination of TMD and CT sequences to direct foreign proteins to seed PSVs via different pathways, referring to FIG. 1 and FIG. 2.

FIG. 1 and FIG. 2 show expression cassettes for delivery of soluble proteins to specific PSV subcompartments in transgenic seeds. Chimeric constructs for targeting recombinant proteins to (A) PSV globoid subcompartment via Golgi and (B) PSV crystalloid subcompartment from ER directly (bypassing the Golgi, which would avoid plant Golgi-specific modifications including N-linked glycosylation). Phaseolin is a seed-specific promoter that allows a high level of expression in transgenic seeds (Altenbach, S. B., et al. (1989), Enhancement of the methionine content of seed protein by the expression of a chimeric gene encoding a methionine-rich protein in transgenic plants, Plant Mol. Biol. 13, 513-522) and Nos is a 3′ terminator. The TMD sequences in (A) and (B) are both derived from BP-80, and the CT sequences in (A) and (B) are derived from BP-80 and α-TIP, respectively (Wink, M. (1993), The plant vacuole: a multifunctional compartment, J. Exp. Bot. 44, 231-146; Raikhel, N. V. and Vitale, A. (1999), What do proteins need to reach different vacuoles? Trends Plant Sci. 4, 149-155). The coding sequences for the recombinant protein with a signal peptide are cloned in frame between the BamHI and EcoRI sites. The figure is not drawn to scale. Abbreviations and amino acid sequences (underlined sequences being derived from BP-80): CT, cytoplasmic tail sequences from either BP-80 (KYRIRQYMDSEIRAIMAQYMPLDSQEEGPNHV) or α-TIP (KYRIRPIEPPPHHHQPLATEDY); PSV, protein storage vacuole; S, spacer (e.g. DYKDDDDKSKTASQAK or other proteolytic cleavage sequence); sp, signal peptide sequences (e.g. MAHARVLLLALAVLATAAVAVA from proaleurain); TGA; TIP, tonoplast, intrinsic protein; TMD, transmembrane domain sequences from BP-80 (TWAAFWVVLIALAMIAGGGFLVY).

In general, the coding sequences of a foreign recombinant protein could be optionally linked to an appropriately selected promoter sequence and TMD or CT encoding sequences. For example, it can be inserted between the BamHI and EcoRI sites as shown in FIG. 1 and FIG. 2. An engineered signal peptide sequence is required if the recombinant protein does not contain a predicted signal sequence that functions in plant cells. A spacer sequence in front of the TMD sequence is included so that the membrane anchor does not affect the structure of the protein and proper protein folding can occur. The fusion protein in FIG. 1 contains the BP-80, TMD and CT sequences, which would direct the foreign protein to the PSV globoid in transgenic seeds via the Golgi (Jiang, L. et al., 2000, Jiang, L. and Rogers, J. C., 1998). By contrast, the fusion protein in FIG. 2 contains the BP-80 TMD and the α-TIP CT sequences, which would direct the foreign protein to the PSV crystalloid via a direct ER-PSV pathway (Jiang, L. et al. (2000), Biogenesis of the protein storage vacuole crystalloid, J Cell Biol. 150, 755-769; Jiang, L. and Rogers, J. C. (1998), Integral membrane protein sorting to vacuoles in plant cells: evidence for two pathways, J. Cell Biol. 143, 1183-1199). The α-TIP CT sequence would be particularly useful in bypassing the Golgi functions that generate complex N-glycans, which are highly immunogenic in animals. Preferably, these fusion proteins are expressed under the control of an appropriately selected promoter such as the seed-specific phaseolin promoter, which would allow the expression of the foreign proteins exclusively in seeds (Sun, S. S. M. et al. (1981), Intervening sequence in a plant gene: comparison of the partial sequence of cDNA and genomic DNA of French bean phaseolin, Nature 289, 37-41; Slightom, J. S. et al. (1983), Complete nucleotide sequence of a French bean storage protein gene: phaseolin, Proc. Natl. Acad. Sci. U.S.A. 80, 1897-1901).

Results obtained from both in vitro and in vivo expression studies have been consistent with the conclusion that these unique TMD and CT sequences can specifically direct a reporter protein to a defined vacuolar compartment. The TMD and CT delivery systems were adopted as an example serving as anchors for delivering recombinant target proteins and such delivery systems is applicable to other plant seed expression systems as homologues of BP-80 and TIP proteins have been found among several other plant species, including pea, tomato, soybean, tobacco and Arabidopsis. Therefore, even without knowledge of targeting mechanisms in such plant cells, attachments of these soluble proteins to membrane anchors would deliver them to specific vacuolar compartments in transgenic seeds. For example, the BP-80 TMD and CT sequences could be used to target proteases or proteins resistant to an acidified environment to the PSV globoid or to vacuoles in vegetative tissues, whereas the α-TIP CT sequences could be used to deliver other proteins to the PSVs that would favor their stable accumulation. However, care should be taken to ensure that the membrane anchor does not affect the proper folding and the topology of the expressed protein.

3) Plant Transformation and Regeneration

Different type of plant species, including monocots and dicots, and various transformation techniques can be adopted for the present invention. However, it is preferred to use a plant that can be transformed with high transformation efficiency. Expression vectors containing the target protein expression cassettes can be introduced into plants according to known techniques such as Agrobacterium-mediated plant transformation, vacuum infiltration, gene transfer into pollen or calli or protoplast transformation (Bechtold, N., Ellis, J. and Pelletier, G. 1993, In planta Agrobacterium-mediated gene transfer by infiltration of adult Arabidopsis thaliana plants, C. R. Acad. Sci. Paris, Life Sci. 316, 1194-1199; Fisher, D. K. and Guiltinan, M. J. 1995, Rapid, efficient production of homozygous transgenic tobacco plants with Agrobacterium tumefaciens: A seed-to-seed protocol, Plant Mol. Biol. 13, 278-289). An ordinary skilled person in the art can make use of different strains of bacteria and transformation methods for the transformation of different host plants according to these known techniques.

Plant regeneration is well known in the art. Transformants screened for desirable gene products were used for regeneration. The regenerated shoots (leaf-disc technique) or green plants (vacuum infiltration) were transferred in soil and grown in green house for further expression analysis.

### EXAMPLE 1

### Expression Cassettes for Target Proteins Expression

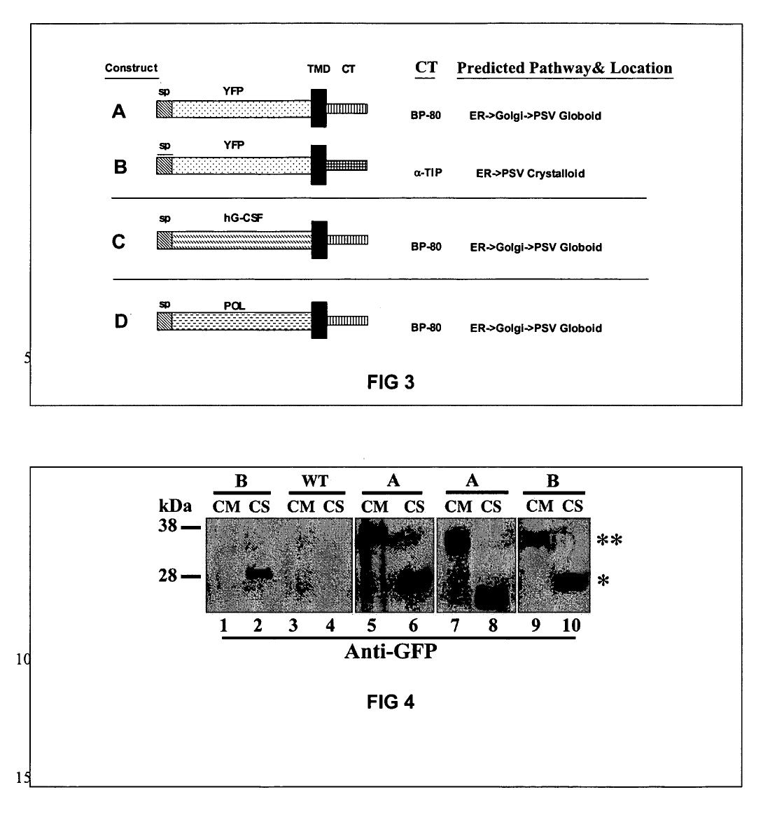

To illustrate that unique membrane anchors can deliver proteins of different origins to the protein storage vacuoles, we used three proteins as reporters: a yellow fluorescent protein (YFP) that can be detected via auto-fluorescent or anti-GFP (green fluorescent protein) antibody, a hG-CSF (human granulocyte-colony stimulating factor) protein, and a POL (Polygonatum odoratum lectin) protein in four expression cassettes. These three proteins were fused at the N-terminal of transmembrane domain (TMD) sequences of BP-80 and the cytoplasmic tail (CT) sequences from either BP-80 (constructs A, C and D) or the alpha-TIP (tonoplast intrinsic protein) (construct B).YFP was fused to constructs A and B, and hG-CSF and POL proteins were fused to C and D, respectively. In addition, the signal peptide sequences (sp) from the barley cysteine protease aleurain (MAHARVLLLALAVLATAAVAVA) or from the rice storage protein glutelin (MASINRPIVFFTVCLFLLCDGSLA) were included at the N-terminal of the reporter fusion proteins. The resulting fusions were then placed under the control of either the 35S CaMV promoter (constructs A and B) or the seed-specific glutelin Gt1 promoter (constructs C and D) and the Nos 3′ terminator. FIG. 3 shows the schematic diagrams of the four expression cassettes constructs used in this invention with information on origins of specific sequences and predicted subcellular localization/pathways. Towards this goal, transgenic tobacco plants expressing construct A or B, and transgenic rice expressing construct C or D have been generated for subsequent analysis of the target proteins expression.

### EXAMPLE 2

### Proteins Expression in Plant Leaves

The two constructs A and B (FIG. 1) generated in EXAMPLE 1 were transformed into tobacco via Agrobacterium-mediated transformation and transgenic kanamycin-resistant tobacco plants were then regenerated and grown in green house. Using Western blot analysis with anti-GFP antibody, we successfully demonstrated target proteins expression in leaves of transgenic plants (FIG. 4). Both soluble (CS) and membrane (CM) proteins were extracted from transgenic plants expressing either construct A (lanes 5-8) or construct B (lanes 1-2 and 9-10) and from wild type (WT) control plant (lanes 5-6). As shown in FIG. 4, the full-length membrane reporter protein with the right expected size was detected only in the CM fraction from plants expressing constructs A or B (lanes 1, 5, 7 and 9; double asterisks). In addition, a protein with the same size as YFP was also detected in the CS fractions from plants expressing constructs A or B (lanes 2, 6, 8 and 10; single asterisk), a result indicating that the YFP was cleaved from the TMD/CT sequences. No signal was detected from wild type plant (lanes 3-4).

### EXAMPLE 3

### Protein Expression in Plant Seeds and Targeting to Protein Storage Vacuoles

In this example, we extracted proteins (both soluble and membrane) from transgenic seeds expressing construct A or B, followed by analysis via SDS-PAGE and Western blot detection with an anti-GFP antibody. As shown in FIG. 5, only cleaved soluble YFP proteins were detected in seeds expressing the constructs (lanes 3, 5, 7, single asterisk). Therefore, it demonstrated that through the application of unique TMD/CT sequences, we successfully directed the YFP reporter protein to the seed protein storage vacuoles of transgenic tobacco, where the YFP protein was separated from the membrane anchors.

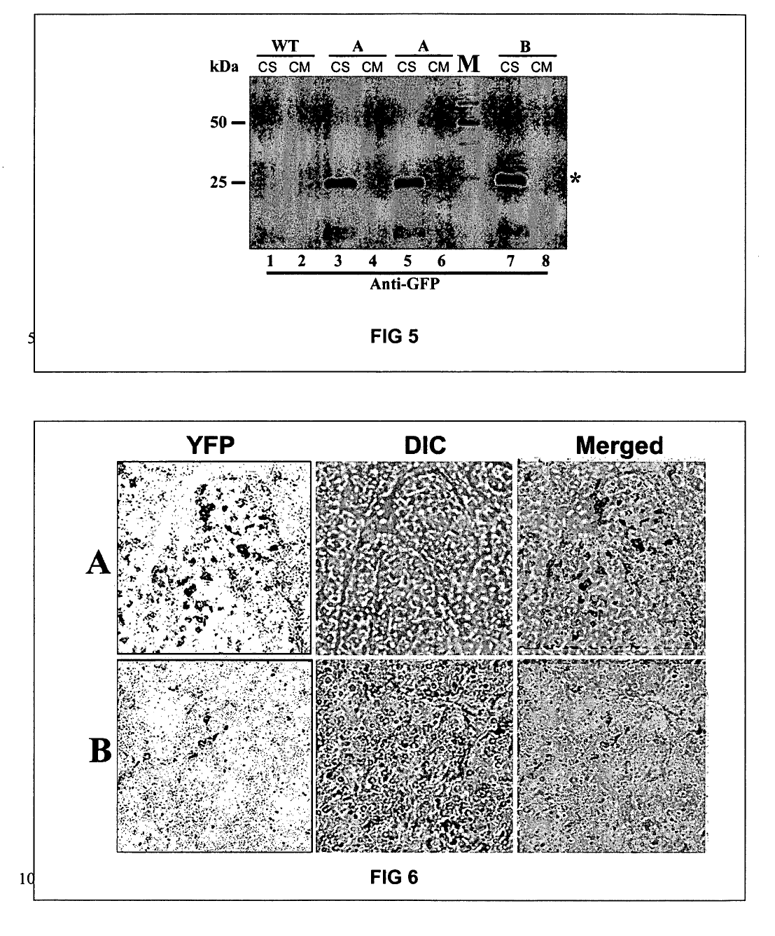

We further studied that subcellular localization of the YFP fusion proteins in transgenic seeds as we prepared fresh sections from transgenic developing seeds (16 days after pollination) expressing construct A or B and observed fluorescent signals directly using confocal laser scanning microscope. As shown in FIG. 6, YFP signals were detected within the protein storage vacuoles of transgenic seeds expressing either construct. Furthermore, the YFP signal patterns in seeds expressing construct A were different from those expressing construct B, indicating that these two fusion proteins may locate to distinct subcompartments of seed protein storage vacuoles.

### EXAMPLE 4

### Expression and Targeting of Proteins of Various Origins in Different Plant Species

Apart from using the reporter protein YFP in EXAMPLES 2 and 3, we further proved that the delivery system in this invention also works in other plant species and for other proteins by adopting other two reporter proteins namely the hG-CSF and POL for the transformation of another plant species, the rice.

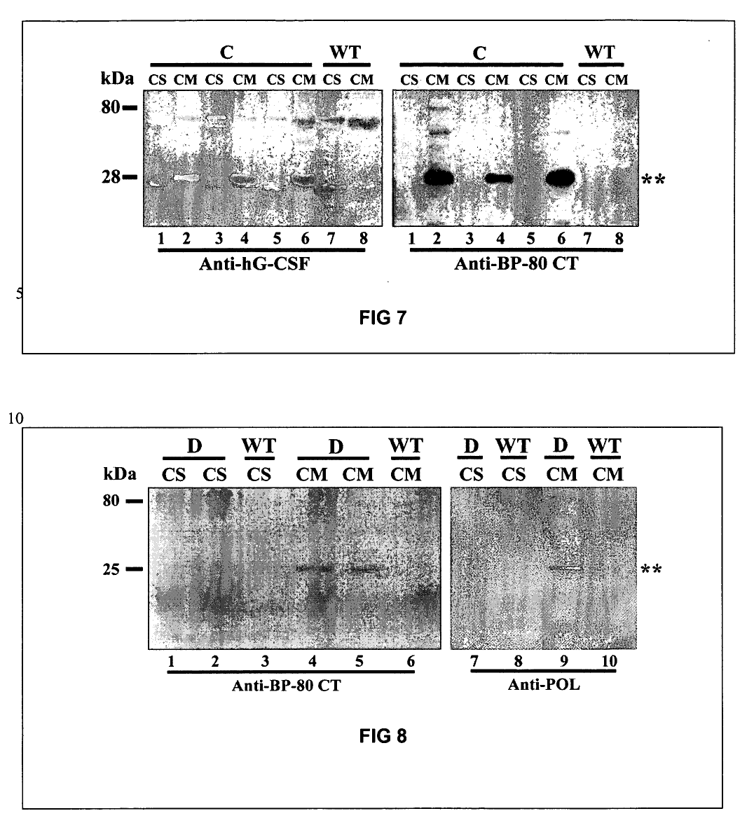

We generated transgenic rice expressing construct C (from EXMAPLE 1) under the control of the Gt1 seed-specific promoter for further analysis. Similarly, both soluble and membrane proteins were extracted from mature seeds of three individual transgenic plants, followed by SDS-PAGE and Western blot analysis. As shown in FIG. 7, the full-length hG-CSF fusion with a correct expected size was detected only in the membrane fractions of transgenic seeds when anti-hG-CSF antibodies were used (left panel, lanes 2, 4 and 6; double asterisks). Moreover, when another identical set of protein samples was detected using antibodies that recognize the BP-80 CT, the same full-length fusion protein was detected in the membrane fractions of transgenic seeds (right panel, lanes 2, 4 and 6; double asterisks). Again, no such fusion protein was detected in wild type seeds.

The system flexibility is further proved by transferring POL fusion (construct D from EXMAPLE 1) into rice via Agrobacterium-mediated transformation. Mature seeds obtained from transgenic rice were further analyzed for the expressed proteins. As shown in FIG. 8, the intact POL fusion protein with an expected size was detected in the membrane fractions of transgenic seeds (lanes 4, 5 and 9; double asterisks) when either BP-80 CT or POL antibodies were used in Western blot detection. Again, no signal was detected from wild type seeds (lanes 3, 6, 8 and 10).

Our data thus far also indicated that the processing of the reporter fusion proteins in transgenic tobacco seeds is different from that in rice seeds because only soluble YFP was detected in tobacco seeds while intact full-length membrane reporter protein was detected in rice seeds. Provided that the fusion protein reaches the protein storage vacuoles in seeds of both tobacco and rice as predicted, it is thus possible that their internal environment may be different such that tobacco seed protein storage vacuoles may contain distinct proteases responsible for the processing of the reporter. The fact that the reporter protein was separated from the membrane anchor upon reaching the protein storage vacuole will be a great advantage for downstream processing in which the targeted proteins can be enriched and purified easily.

The method can be applied to production of any target proteins from different origins to be produced in a considerable amount through properly selecting the seed specific promoter in the way that the target protein encoding sequence insert can be highly transcribed in the transformed plant seeds. By fusing or inserting the target protein encoding sequence with appropriate transmembrane domain and cytoplasmic tail sequence serving as anchors, recombinant target proteins can be delivered via distinct vesicular transport pathways to specific vacuolar compartments in such a way that it may also provide flexibility for target proteins to acquire post-translational modifications. The target proteins can then be cleaved, recovered and purified for their nutritional values or biological activities. The present invention thereby provides the method for enhanced and stable production of target proteins in transgenic plant seeds which can be consumed as food by human or animals. The examples are offered by way of illustration and should not be interpreted as limitation on the scope of the invention.

We claim:

1. A DNA construct to generate and direct the processing, targeting and stably accumulating of target proteins in transgenic plant seeds, comprising:
a promoter sequence capable of directing expression in cells of the plant seeds;
a first DNA sequence encoding the target proteins;
a second DNA sequence having a transmembrane domain sequence and a cytoplasmic tail sequence which serve as anchors for delivering the target proteins to subcompartments of protein storage vacuoles of the cells; and
a third DNA sequence functioning as a termination region in the plant.

2. The DNA construct of claim 1, wherein the promoter is a seed-specific promoter.

3. The DNA construct of claim 2, wherein the promoter comprises a phaseolin promoter or a glutelin Gt1 promoter.

4. The DNA construct of claim 1, wherein the transmembrane domain sequence is derived from BP-80 and the cytoplasmic tail sequence is derived from BP-80 or α-TIP.

5. The DNA construct of claim 2, wherein the transmembrane domain sequence is derived from BP-80 and the cytoplasmic tail sequence is derived from BP-80 or α-TIP.

6. The DNA construct of claim 3, wherein the transmembrane domain sequence is derived from BP-80 and the cytoplasmic tail sequence is derived from BP-80 or α-TIP.

7. The DNA construct of the claim 1, wherein the subcompartments comprise globoids or crystalloids.

8. The DNA construct of claim 1, wherein the third DNA sequence is an NOS terminator.

9. The DNA construct of claim 1, further comprising a spacer sequence in front of the transmembrane domain sequence so that the anchor does not affect proper folding of the target protein.

10. The DNA construct of claim 2, further comprising a spacer sequence in front of the transmembrane domain sequence so that the anchor does not affect proper folding of the target protein.

11. The DNA construct of claim 9, wherein the spacer sequence is a proteolytic cleavage sequence.

12. The DNA construct of claim 10, wherein the spacer sequence is a proteolytic cleavage sequence.

13. The DNA construct of claim 11, wherein the protein storage vacuoles and their subcompartments provide a protease activity acting with the proteolytic cleavage sequence so that the target protein separates from the transmembrane domain.

14. The DNA construct of claim 12, wherein the protein storage vacuoles and their subcompartments provide a protease activity acting with the proteolytic cleavage sequence so that the target protein separates from the transmembrane domain.

15. The DNA construct of claim 10, further comprising an engineered signal peptide sequence.

16. The DNA construct of claim 15, wherein the signal peptide sequence is derived from proaleurain.

17. A vector comprising a DNA construct as defined in claim 1.

18. A host cell comprising a vector as defined in claim 17.

19. The host cell of claim 18, wherein the host cell is a plant cell.

20. The host cell of claim 19, wherein the plant cell is a monocot cell or a dicot cell.

21. A transgenic plant or progeny thereof comprising a DNA construct as defined in claim 1.

22. A transgenic plant seed comprising a DNA construct as defined in claim 1.

23. A method to construct a transgenic plant, comprising the steps of:
a) constructing a vector including a DNA construct defined as in claim 1;
b) transforming plant cells with the vector; and
c) regenerating the transgenic plant from the plant cells to produce the target proteins in seeds of the transgenic plant.

24. The method of claim 23, wherein the vector is a plasmid vector.

25. The method of claim 24, wherein the plasmid vector is a binary or superbinary vector.

26. The method of claim 25, wherein the vector is pSB130 or pBI121.

27. The method of claim 23, wherein the plant is tobacco or rice.

28. A method claim 27, wherein the plant cells are transformed utilizing an Agrobacterium system.

29. A method of claim 28, wherein the Agrobacterium system is an Agrobacterium tumefaciens-Ti plasmid system.
